(19) United States
(12) Patent Application Publication
(10) Pub. No.: US 2013/0289407 A1
(43) Pub. Date: Oct. 31, 2013

(54) 3D ULTRASOUND SYSTEM FOR EXTENDING VIEW OF IMAGE AND METHOD FOR OPERATING THE 3D ULTRASOUND SYSTEM

(71) Applicant: SAMSUNG MEDISON CO., LTD., (US)

(72) Inventor: KWANG-HEE LEE, GANGWON-DO (KR)

(73) Assignee: SAMSUNG MEDISON CO., LTD., GANGWON-DO (KR)

(21) Appl. No.: 13/844,573

(22) Filed: Mar. 15, 2013

Related U.S. Application Data
(63) Continuation-in-part of application No. 13/230,352, filed on Sep. 12, 2011.

(30) Foreign Application Priority Data
Sep. 14, 2010 (KR) 10-2010-0090122
Oct. 11, 2012 (KR) 10-2012-0113036

(51) Int. Cl. A61B 8/14

(52) U.S. Cl. CPC A61B 8/145
USPC 600/443

(57) ABSTRACT

A method of operating a 3D ultrasound system, the method including generating a first volume image and a second volume image of an object by using ultrasound signals; determining a first anatomical plane and at least one second anatomical plane that is different from the first anatomical plane from each of the first volume image and the second volume image; matching polygonal templates to the at least one second anatomical plane of each of the first volume image and the second volume image; and overlapping axis of the first anatomical planes of the first volume image and the second volume image and overlapping the first volume image and the second volume image based on energy of a polygonal template matched to the at least one second anatomical plane of the first volume image and energy of a polygonal template matched to the at least one second anatomical plane of the second volume image.

### REFERENCE TO RELATED PATENT APPLICATION

This application is a continuation-in-part application which claims the benefit of U.S. patent application Ser. No. 13/230,352, filed on Sep. 12, 2011, in the USPTO, the disclosure of which is incorporated herein in its entirety by reference.

### BACKGROUND OF THE INVENTION

1. Field of the Invention

The present invention relates to a 3D ultrasound system for extending view of image with respect to an object by generating a plurality of volume images by using ultrasound signals and overlapping the generated volume images and a method for operating the 3D ultrasound system.

2. Description of the Related Art

An ultrasound system is a device for transmitting ultrasound signals from the surface of a human body toward a predetermined region inside the human body (that is, an object, such as a fetus or an internal organ) and acquiring images of a tomogram of a soft-tissue or blood flow by using data regarding an ultrasound signal reflected by a tissue inside the human body.

Such an ultrasound system features small size, low cost, real-time display, and no radiation exposure (e.g., X-rays). Therefore, ultrasound systems are widely used with other types of imaging systems, such as an X-ray system, a computerized tomography (CT) scanner, a magnetic resonance image (MRI) system, a nuclear medicine system, etc.

An ultrasound system may generate a plurality of volume images by switching scanning regions of an object according to a particular part of the object to be viewed.

Therefore, a technique for an image with an extended view by providing a single image by overlapping a plurality of volume images acquired by scanning different regions of an object is in demand.

### SUMMARY OF THE INVENTION

Exemplary embodiments address at least the above problems and/or disadvantages and other advantages not described above. Also, an exemplary embodiment is not required to overcome the disadvantages described above, and an exemplary embodiment may not overcome any of the problems described above.

One or more exemplary embodiments provide a method of extending view of an image by generating a plurality of volume images with respect to an object by using ultrasound signals and overlapping the plurality of images by using the energy of a polygon template corresponding to the volume images.

According to an aspect of an exemplary embodiment, there is provided a method of operating a 3D ultrasound system, the method comprising: generating a first volume image and a second volume image of an object by using ultrasound signals; determining a first anatomical plane and at least one second anatomical plane that is different from the first anatomical plane from each of the first volume image and the second volume image; matching polygonal templates to the at least one second anatomical plane of each of the first volume image and the second volume image; and overlapping axis of the first anatomical planes of the first volume image and the second volume image and overlapping the first volume image and the second volume image based on energy of a polygonal template matched to the at least one second anatomical plane of the first volume image and energy of a polygonal template matched to the at least one second anatomical plane of the second volume image.

The overlapping of the first volume image and the second volume image may comprise overlapping the first volume image and the second volume image based on a second anatomical plane of the first volume image and a second anatomical plane of the second volume image corresponding to a case in which a difference between energy of a polygonal template is within a predetermined range.

The overlapping of the first volume image and the second volume image may comprise overlapping the first volume image and the second volume image based on a second anatomical plane corresponding to the smallest energy of a polygonal template from among at least one second anatomical plane of the first volume image and a second anatomical plane corresponding to the smallest energy of a polygonal template from among at least one second anatomical plane of the second volume image.

The first anatomical plane may comprise a sagittal plane, a coronal plane, or a transverse plane.

The first anatomical plane may be perpendicular to the at least one second anatomical plane.

The method may further comprise rendering the overlapped first volume image and the second volume image to a single image and displaying the single image.

According to another aspect of an exemplary embodiment, there is provided a 3D ultrasound system comprising: a scanning unit which generates a first volume image and a second volume image of an object by using ultrasound signals; a processor which determines a first anatomical plane and at least one second anatomical plane that is different from the first anatomical plane from each of the first volume image and the second volume image; a matching unit which matches polygonal templates to the at least one second anatomical plane of each of the first volume image and the second volume image; and a control unit which overlaps axis of the first anatomical planes of the first volume image and the second volume image and overlaps the first volume image and the second volume image based on energy of a polygonal template matched to the at least one second anatomical plane of the first volume image and energy of a polygonal template matched to the at least one second anatomical plane of the second volume image.

The control unit may overlap the first volume image and the second volume image based on a second anatomical plane of the first volume image and a second anatomical plane of the second volume image corresponding to a case in which a difference between energy of a polygonal template is within a predetermined range.

The control unit may overlap the first volume image and the second volume image based on a second anatomical plane corresponding to the smallest energy of a polygonal template from among at least one second anatomical plane of the first volume image and a second anatomical plane corresponding to the smallest energy of a polygonal template from among at least one second anatomical plane of the second volume image.

The first anatomical plane may comprise a sagittal plane, a coronal plane, or a transverse plane.

The first anatomical plane may be perpendicular to the at least one second anatomical plane.

The 3D ultrasound system may further comprise a rendering unit which renders the overlapped first volume image and the second volume image to a single image and displays the single image.

According to another aspect of an exemplary embodiment, there is provided a computer-readable recording medium having recorded thereon a program for executing the method.

### BRIEF DESCRIPTION OF THE DRAWINGS

The above and other features and advantages of the present invention will become more apparent by describing in detail exemplary embodiments thereof with reference to the attached drawings in which:

FIG. 1 is a diagram of a 3D ultrasound system 101 according to an embodiment of the present invention;

FIG. 2 is a diagram showing an example of overlapping volume images in a 3D ultrasound system according to an embodiment of the present invention;

FIG. 3A and FIG. 3B are diagrams showing overlapping volume images in a 3D ultrasound system according to another embodiment of the present invention;

FIG. 4 is a flowchart showing a method of operating a 3D ultrasound system, according to an embodiment of the present invention;

FIG. 5 is a flowchart showing a method of operating a 3D ultrasound system, according to another embodiment of the present invention.

### DETAILED DESCRIPTION OF THE INVENTION

Hereinafter, the present invention will be described in detail by explaining preferred embodiments of the invention with reference to the attached drawings. Like reference numerals in the drawings denote like elements.

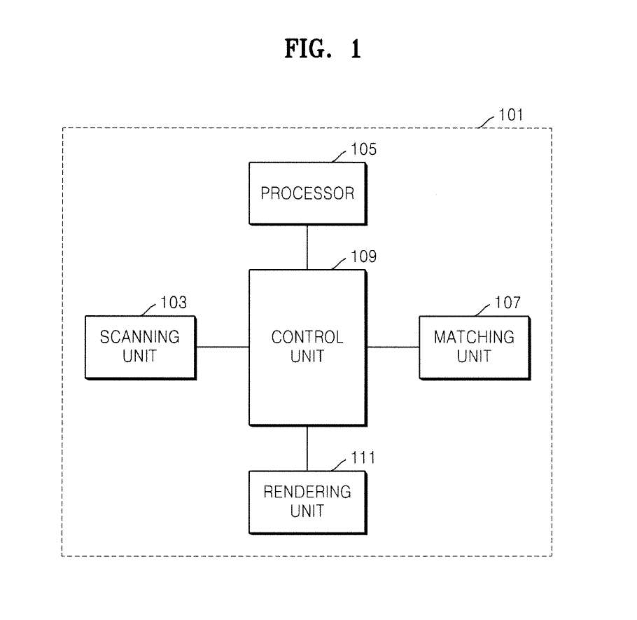

FIG. 1 is a diagram of a 3D ultrasound system 101 according to an embodiment of the present invention.

Referring to FIG. 1, the 3D ultrasound system 101 includes a scanning unit 103, a processor 105, a matching unit 107, a control unit 109, and a rendering unit 111.

The scanning unit 103 generates a plurality of volume images with respect to an object by using ultrasound signals. Here, the scanning unit 103 may generate a plurality of volume images by switching scanning regions of the object to be scanned.

The processor 105 is input a landmark related to the object, identifies a first point corresponding to the landmark in a first volume image from among the plurality of volume images, and identifies a second point corresponding to the landmark in a second volume image from among the plurality of volume images. For example, the processor 105 may be input “thalamus” as the landmark and may set up seeds at first and second points corresponding to the thalamus.

There, since the first and second volume images include a particular same region of an object and different regions of the object, a landmark may be input in relation to the particular region of the object. For example, in a case where an object is the head of a fetus and both the first and second volume images include the head of the fetus, the processor 105 may be input a landmark related to the head of the fetus.

The control unit 109 overlaps the first and second volume images from among the plurality of volume images.

In detail, the control unit 109 may overlap the first and second volume images based on a first point corresponding to the landmark with respect to the first image and a second point corresponding to the landmark with respect to the second image.

Here, the control unit 109 may rotate the first and second volume images, compare a first coordinate of the first point in the first volume image and a second coordinate of the second point in the second volume image, and overlap the first and second volume images at a rotated position at which the first and second points overlap in a permissible range as a result of the comparison. Here, the narrower the permissible range is (that is, the smaller the distance between the first coordinate and the second coordinate is), the control unit 109 may overlap the first and second volume images with better synchronization with the object.

Furthermore, if n landmarks (n is a natural number equal to or greater than 3) are input, the control unit 109 may rotate the first and second volume images, compare a first symbol which connects the first points in the first volume image and a second symbol which connects the second points in the second volume image, and overlap the first and second volume images at a rotated position at which the first and second symbols overlap in a permissible range as a result of the comparison.

The control unit 109 may better represent the object by overlapping the first and second volume images by using the first symbol in the first volume image and the second symbol in the second volume image that are related to the n landmarks.

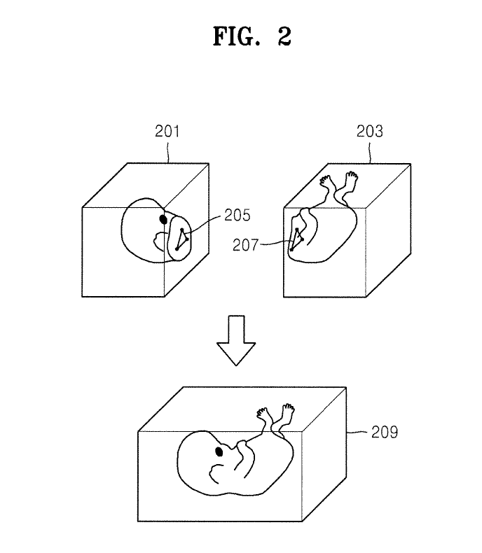

FIG. 2 is a diagram showing an example of overlapping volume images in a 3D ultrasound system according to an embodiment of the present invention.

Referring to FIG. 2, the 3D ultrasound system may generate a plurality of volume images with respect to an object by using ultrasound signals and may be input n landmarks (n is a natural number equal to or greater than 3) regarding the object. The 3D ultrasound system may rotate each of the first and second volume images from among the plurality of generated volume images, compare a first symbol which connects first points corresponding to the landmarks in the first volume image and a second symbol which connects the second points corresponding to the landmarks in the second volume image, overlap the first and second volume images at a rotated position at which the first and second symbols overlap in a permissible range as a result of the comparison, and display the overlapping first and second volume images as a single image.

For example, the 3D ultrasound system may generate a first volume image 201 and a second volume image 203 with respect to a fetus by using ultrasound signals, be input 3 landmarks regarding the center of the fetus, rotate each of the first volume image 201 and the second volume image 203, and overlap the first volume image 201 and the second volume image 203 at a rotated position at which a first symbol 205 which connects first points corresponding to the landmarks in the first volume image 201 and a second symbol 207 which connects second points corresponding to the landmarks in the second volume image 203 overlap, and display the overlapped first and second volume 201 and 203 as a single image 209.

Meanwhile, the scanning unit 103 shown in FIG. 1 may generate a first volume image and a second volume image regarding the object by using ultrasound signals.

The processor 105 may determine a first anatomical plane and at least one second anatomical plane that is different from the first anatomical plane from each of the first volume image and the second volume image. The first anatomical plane may include a sagittal plane, a coronal plane, or a transverse plane, and may be perpendicular to the at least one second anatomical plane.

The matching unit 107 may match a polygonal template with respect to at least one second anatomical plane of each of the first volume image and the second volume image. In detail, the matching unit 107 may match a polygonal template similar to shape of a cross-sectional image of the at least one second anatomical plane to the corresponding second anatomical plane. The shapes of the polygonal templates matched to the at least one second anatomical plane of the first volume image and the at least one second anatomical plane of the second volume image may be the same each other.

The control unit 109 overlaps the axis of first anatomical planes of the first volume image and the second volume image and may overlap the first volume image and the second volume image based on energy of the polygonal template matched to the at least one second anatomical plane of the first volume image and energy of the polygonal template matched to the at least one second anatomical plane of the second volume image.

Energy of a polygonal template may be measured by using a distance map regarding a cross-sectional image, for example. The distance map includes the shortest distances from respective pixels included in a cross-sectional image to pixels at a boundary of the cross-sectional image. When a polygonal template is matched to a cross-sectional image, energy of the polygonal template may be measured by using distances of pixels to which the polygonal template is applied. Since distances of pixels at a boundary of the cross-sectional image are zero, if the polygonal template is precisely matched to the cross-sectional image, sum of distances of the pixels to which the polygonal template is applied will be zero. In other words, the smaller the sum of distances of pixels of a cross-sectional image to which a polygonal template is applied, the more precisely the polygonal template is matched to the cross-sectional image.

When a difference between energy of a polygonal template matched to a second anatomical plane of the first volume image and energy of a polygonal template matched to a second anatomical plane of the second volume image is within a predetermined range, the control unit 109 may overlap the first volume image and the second volume image based on the second anatomical plane of the first volume image and the second anatomical plane of the second volume image

From among at least one second anatomical plane of the first volume image and at least one second anatomical plane of the second volume image, second anatomical planes to which polygonal templates having a same energy are matched may indicate that the corresponding second anatomical planes are the same cross-sectional images. Therefore, the control unit 109 determines a single second anatomical plane from the first volume image and a single second anatomical plane from the second volume image corresponding to polygonal templates having energies of which a difference is within a predetermined range and overlaps the first volume image and the second volume image based on locations of the single second anatomical plane of the first volume image and the single second anatomical plane of the second volume image.

Furthermore, the control unit 109 may overlap the first volume image and the second volume image based on a second anatomical plane corresponding to the smallest energy of the polygonal template matched to the at least one second anatomical plane of the first volume image and a second anatomical plane corresponding to the smallest energy of the polygonal template matched to the at least one second anatomical plane of the second volume image.

As described above, the smallest energy of a polygonal template indicates that shape of the polygonal template is very similar to shape of a second anatomical plane. Therefore, the control unit 109 may determine a second anatomical plane corresponding to the smallest energy of a polygonal template from among at least one second anatomical plane of the first volume image and a second anatomical plane corresponding to the smallest energy of a polygonal template from among at least one second anatomical plane of the second volume image and may overlap the first volume image and the second volume image based on locations of the second anatomical plane of the first volume image and the second anatomical plane of the second volume image.

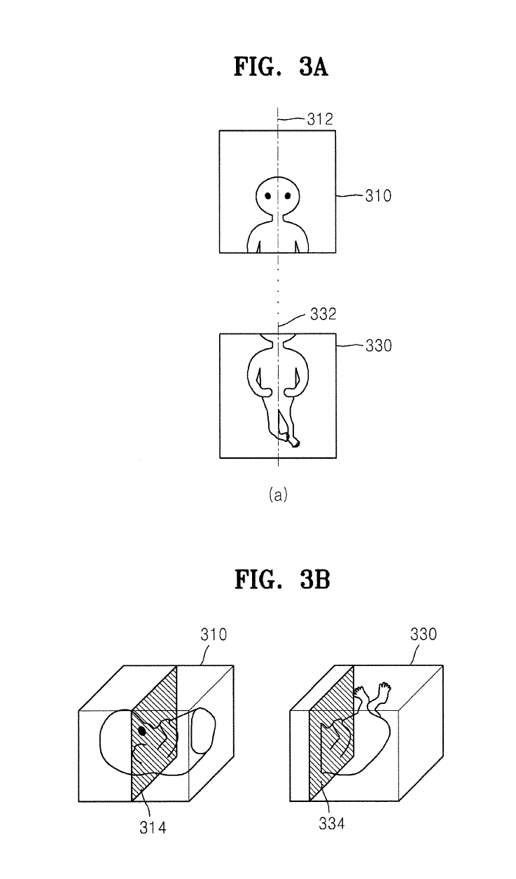

FIG. 3 is a diagram showing overlapping volume images in a 3D ultrasound system according to another embodiment of the present invention.

FIG. 3 shows a first volume image 310 and a second volume image 330 regarding a fetus, where the first volume image 310 and the second volume image 330 are partially identical to each other. A 3D ultrasound system 101 according to an embodiment of the present invention may overlap the first volume image 310 and the second volume image 330 at mutually identical portions.

FIG. 3(a) is a diagram showing the first volume image 310 and the second volume image 330 of the fetus, viewed from the top, where the processor 105 may determine sagittal planes of the first volume image 310 and the second volume image 330 as first anatomical planes of the same. The sagittal planes may be mid-sagittal planes of the first volume image 310 and the second volume image 330.

The control unit 109 may overlap the vertical axis of the first volume image 310 and the vertical axis of the second volume image 330 by overlapping axis 312 and 332 of the sagittal planes of the first volume image 310 and the second volume image 330.

Referring to FIG. 3(b), the processor 105 may obtain at least one transverse plane as the second anatomical plane from each of the first volume image 310 and the second volume image 330. An elliptical template may be matched to the at least one transverse plane by the matching unit 107. In detail, the matching unit 107 may move, rotate, and deform an elliptical template having a predetermined shape and match the elliptical template to the cross-sectional image of the at least one transverse plane. The matching unit 107 may deform the elliptical template by changing length of the long axis, length of the short axis, perimeter, or a ratio between the length of the long axis and the length of the short axis.

As described above, the control unit 109 may determine a transverse plane 314 of the first volume image 310 and a transverse plane 334 of the second volume image 330 corresponding to a case in which a difference between energy of a polygonal template matched to at least one transverse plane of the first volume image 310 and energy of a polygonal template matched to at least one transverse plane of the second volume image 330 is within a predetermined range and may overlap thte first volume image 310 and the second volume image 330 at the determined planes 314 and 334. Therefore, the horizontal axis of the first volume image 310 overlaps the horizontal axis of the second volume image 330.

Furthermore, the control unit 109 may determine the transverse plane 314 corresponding to the smallest energy of a polygonal template from among at least one transverse planes of the first volume image 310 and the transverse plane 334 corresponding to the smallest energy of a polygonal template from among at least one transverse planes of the second volume image 330 and may overlap the first volume image 310 and the second volume image 330 at the determined planes 314 and 334.

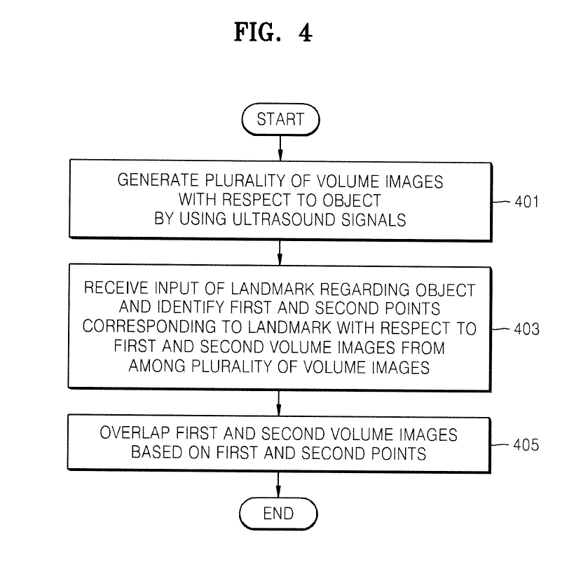

FIG. 4 is a flowchart showing a method of operating a 3D ultrasound system, according to an embodiment of the present invention.

Referring to FIG. 4, in operation 401, the 3D ultrasound system generates a plurality of volume images with respect to an object by using ultrasound signals. Here, the 3D ultrasound system may generate the plurality of volume images by switching scanning regions of the object to be scanned.

In operation 403, the 3D ultrasound system is input a landmark regarding the object, identifies a first point corresponding to the landmark in a first volume image from among the plurality of images, and identifies a second point corresponding to the landmark in a second volume image from among the plurality of images.

Here, since the first and second volume images include a particular same region of an object and different regions of the object, the 3D ultrasound system may be input a landmark in relation to the particular region of the object.

In operation 405, the 3D ultrasound system overlaps the first and second volume images based on the first and second points, renders the overlapping first and second volume images into a single image, and displays the single image.

Here, the 3D ultrasound system may rotate the first and second volume images, compare a first coordinate of the first point in the first volume image and a second coordinate at of the second point in the second volume image, and overlap the first and second volume images at a rotated position at which the first and second points overlap in a permissible range as a result of the comparison.

Furthermore, if n landmarks (n is a natural number equal to or greater than 3) are input, the 3D ultrasound system may rotate the first and second volume images, compare a first symbol which connects the first points existing in the first volume image and a second symbol which connects the second points existing in the second volume image, and overlap the first and second volume images at a rotated position at which the first and second symbols overlap in a permissible range as a result of the comparison.

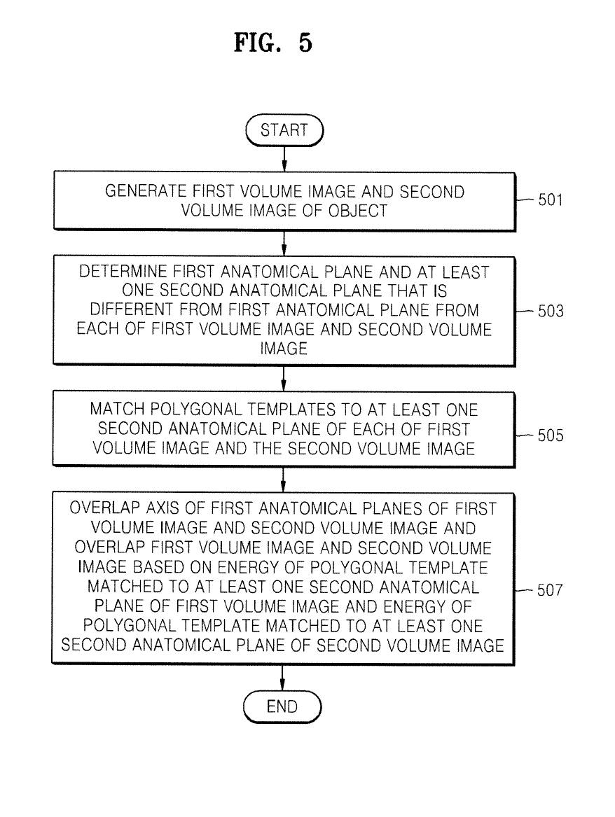

FIG. 5 is a flowchart showing a method of operating a 3D ultrasound system, according to another embodiment of the present invention.

First, in an operation 501, the 3D ultrasound system 101 generates a first volume image and a second volume image of an object.

In an operation 503, the 3D ultrasound system 101 determines a first anatomical plane and at least one second anatomical plane that is different from the first anatomical plane from each of the first volume image and the second volume image.

In an operation 505, the 3D ultrasound system 101 matches polygonal templates to the at least one second anatomical plane of each of the first volume image and the second volume image.

In an operation 507, the 3D ultrasound system 101 overlaps axis of the first anatomical planes of the first volume image and the second volume image and overlaps the first volume image and the second volume image based on energy of a polygonal template matched to the at least one second anatomical plane of the first volume image and energy of a polygonal template matched to the at least one second anatomical plane of the second volume image.

According to an embodiment of the present invention, the 3D ultrasound system may extend the view of an image with respect to an object by generating a plurality of volume images with respect to an object by using ultrasound signals and overlapping the plurality of images by using energy of a polygon template matched to the plurality of volume images.

Embodiments of the present invention provide computer readable recording media having recorded thereon program commands for executing operations in various computers. The computer readable recording media may include program commands, data files, data structures, or combinations thereof. Computer commands recorded in the computer readable recording media may either be designed and configured exclusively for the present invention or be already known and usable in the related art. Examples of the computer readable recording media include magnetic media, such as hard disk drives, floppy disk drives, and magnetic tapes, optical media, such as CD-ROMs, DVDs, etc., magneto-optical media, such as floptical disks, and hardware devices that are specially designed to store and execute program commands, such as ROMs, RAMs, flash memories, etc. Examples of the program codes include not only machine codes written by compilers, but also advanced language codes that may be executed on computers by using interpreters or the like.

While this invention has been particularly shown and described with reference to preferred embodiments thereof, it will be understood by those skilled in the art that various changes in form and details may be made therein without departing from the spirit and scope of the invention as defined by the appended claims. The preferred embodiments should be considered in descriptive sense only and not for purposes of limitation. Therefore, the scope of the invention is defined not by the detailed description of the invention but by the appended claims, and all differences within the scope will be construed as being included in the present invention.

What is claimed is:

1. A method of operating a 3D ultrasound system, the method comprising:
generating a first volume image and a second volume image of an object by using ultrasound signals;
determining a first anatomical plane and at least one second anatomical plane that is different from the first anatomical plane from each of the first volume image and the second volume image;
matching polygonal templates to the at least one second anatomical plane of each of the first volume image and the second volume image; and
overlapping axis of the first anatomical planes of the first volume image and the second volume image and overlapping the first volume image and the second volume image based on energy of a polygonal template matched to the at least one second anatomical plane of the first volume image and energy of a polygonal template matched to the at least one second anatomical plane of the second volume image.

2. The method of claim 1, wherein the overlapping of the first volume image and the second volume image comprises overlapping the first volume image and the second volume image based on a second anatomical plane of the first volume image and a second anatomical plane of the second volume image corresponding to a case in which a difference between energy of a polygonal template is within a predetermined range.

3. The method of claim 1, wherein the overlapping of the first volume image and the second volume image comprises overlapping the first volume image and the second volume image based on a second anatomical plane corresponding to the smallest energy of a polygonal template from among at least one second anatomical plane of the first volume image and a second anatomical plane corresponding to the smallest energy of a polygonal template from among at least one second anatomical plane of the second volume image.

4. The method of claim 1, wherein the first anatomical plane comprises a sagittal plane, a coronal plane, or a transverse plane.

5. The method of claim 1, wherein the first anatomical plane is perpendicular to the at least one second anatomical plane.

6. The method of claim 1, further comprising rendering the overlapped first volume image and the second volume image to a single image and displaying the single image.

7. A non-transitory computer readable recording medium having recorded thereon a computer program for implementing the method of claim 1.

8. A 3D ultrasound system comprising:
a scanning unit which generates a first volume image and a second volume image of an object by using ultrasound signals;
a processor which determines a first anatomical plane and at least one second anatomical plane that is different from the first anatomical plane from each of the first volume image and the second volume image;
a matching unit which matches polygonal templates to the at least one second anatomical plane of each of the first volume image and the second volume image; and
a control unit which overlaps axis of the first anatomical planes of the first volume image and the second volume image and overlaps the first volume image and the second volume image based on energy of a polygonal template matched to the at least one second anatomical plane of the first volume image and energy of a polygonal template matched to the at least one second anatomical plane of the second volume image.

9. The 3D ultrasound system of claim 8, wherein the control unit overlaps the first volume image and the second volume image based on a second anatomical plane of the first volume image and a second anatomical plane of the second volume image corresponding to a case in which a difference between energy of a polygonal template is within a predetermined range.

10. The 3D ultrasound system of claim 8, wherein the control unit overlaps the first volume image and the second volume image based on a second anatomical plane corresponding to the smallest energy of a polygonal template from among at least one second anatomical plane of the first volume image and a second anatomical plane corresponding to the smallest energy of a polygonal template from among at least one second anatomical plane of the second volume image.

11. The 3D ultrasound system of claim 8, wherein the first anatomical plane comprises a sagittal plane, a coronal plane, or a transverse plane.

12. The 3D ultrasound system of claim 8, wherein the first anatomical plane is perpendicular to the at least one second anatomical plane.

13. The 3D ultrasound system of claim 8, further comprising a rendering unit which renders the overlapped first volume image and the second volume image to a single image and displays the single image.
